(19) United States
(12) Patent Application Publication
(10) Pub. No.: US 2017/0069851 A1
(43) Pub. Date: Mar. 9, 2017

(54) ORGANIC PHOTOELECTRIC CONVERSION DEVICE AND SOLID-STATE IMAGING DEVICE

(71) Applicant: Kabushiki Kaisha Toshiba, Minato-ku (JP)

(72) Inventors: Isao TAKASU, Setagaya (JP); Atsushi WADA, Kawasaki (JP); Machiko ITO, Yokohama (JP); Satomi TAGUCHI, Ota (JP); Fumihiko AIGA, Kawasaki (JP)

(73) Assignee: Kabushiki Kaisha Toshiba, Minato-ku (JP)

(21) Appl. No.: 15/235,933

(22) Filed: Aug. 12, 2016

(30) Foreign Application Priority Data
Sep. 9, 2015 (JP) 2015-177506

(51) Int. Cl. H01L 51/00; C07D 471/06; H01L 27/30

(52) U.S. Cl. CPC H01L 51/0072; H01L 27/307; C07D 471/06; H01L 51/4293

(57) ABSTRACT

An organic photoelectric conversion devise of the embodiment includes a charge transport layer comprised of a plurality of isomers containing a compound represented by a following general formula (1) and an enantiomer of the following general formula (1).

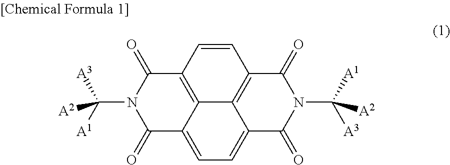

In the general formula (1), A¹, A² and A³ respectively represent a different substituent group.

### CROSS-REFERENCE TO RELATED APPLICATION

This application is based upon and claims the benefit of priority from Japanese Patent Application No. 2015-177506, filed Sep. 9, 2015, the entire contents of which are incorporated herein by reference.

### FIELD

Embodiments of the present invention relate to an organic photoelectric conversion devise and a solid-state imaging device.

### BACKGROUND

An organic photoelectric conversion device used in a solid-state imaging device may be exposed to a high-temperature environment in the production process thereof. However, there were cases where an organic material used for an organic photoelectric conversion device could not sufficiently withstand a high-temperature environment.

Also, it is often that a voltage is applied from the outside to an organic photoelectric conversion device used in a solid-state imaging device in order to improve photoelectric conversion efficiency and response speed. However, a dark current increases due to hole injection or electron injection from an electrode when a voltage is applied from the outside. A dark current causes noise in a solid-state imaging device.

### BRIEF DESCRIPTION OF DRAWINGS

FIG. 1 is a schematic diagram representing the configuration of the organic photoelectric conversion devise of the embodiment.

FIG. 2 is a schematic diagram representing another example of configuration of e organic photoelectric conversion devise of the embodiment.

FIG. 3 is a schematic diagram representing the configuration of the solid-state imaging device of the embodiment.

FIG. 4 is a schematic diagram representing the production method for he solid-state imaging device of the embodiment.

FIG. 5 is a schematic diagram representing the production method for the solid-state imaging device of the embodiment.

FIG. 6 is a schematic diagram representing the production method for the solid-state imaging device of the embodiment.

FIG. 7 is a schematic diagram representing the production method for the solid-state imaging device of the embodiment.

FIG. 8 is a perspective view showing an example of the CMOS image sensor using the solid-state imaging device of the embodiment.

FIG. 9 is a perspective view showing another example of the CMOS image sensor using the solid-state imaging device of the embodiment.

FIG. 10 is a plan view showing an example of the vehicle including the camera equipped with the CMOS image sensor.

FIG. 11 is a plan view showing another example of the vehicle including the camera equipped with the CMOS image sensor.

FIG. 12 is a plan view showing the smartphone including the camera equipped with the CMOS image sensor.

FIG. 13 is a plan view showing the tablet including the camera equipped with the CMOS image sensor.

FIG. 14 is plan and cross-sectional views obtained by taking photographs of a surface of a film, which was formed from compounds having three isomers obtained by reacting sec-butylamine and NTCDA, with a scanning electron microscope (SEM).

FIG. 15 is plan and cross-sectional views obtained by taking photographs of a surface of a film formed from NTCDA alone with a scanning electron microscope (SEM).

FIG. 16 is a schematic diagram for explaining a rotational direction of the molecular structure in the simulation.

FIG. 17 is a schematic diagram for explaining a rotational direction of the molecular structure in the simulation.

### DETAILED DESCRIPTION

The organic photoelectric conversion devise of the embodiment includes a charge transport layer comprised of a plurality of isomers containing the compound represented by the following general formula (1) and an enantiomer of the following general formula (1).

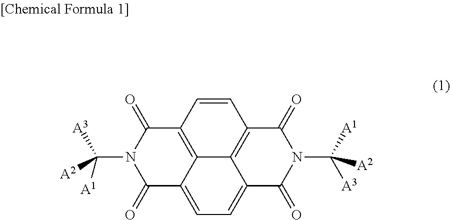

In the general formula (1), A¹, A² and A³ respectively represent a different substituent group.

Hereinafter, the organic photoelectric conversion devise and the solid-state imaging device according to the embodiments are described with reference to the drawings.

### First Embodiment

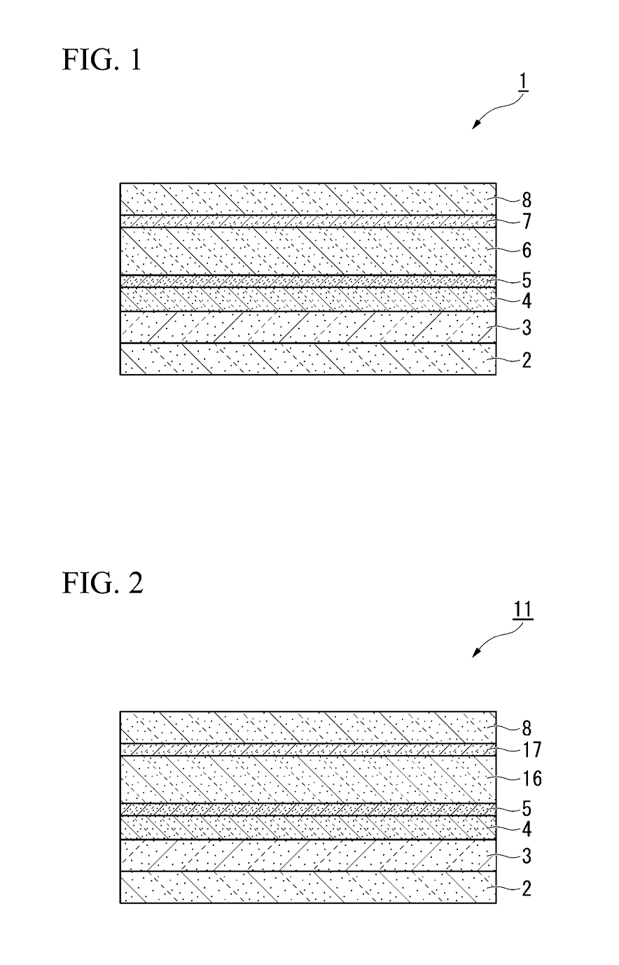

FIG. 1 is a schematic diagram representing the configuration of the organic photoelectric conversion devise of the 1st embodiment. As shown in FIG. 1, the organic photoelectric conversion device 1 includes the substrate 2, the anode 3, the planarization layer 4, the hole transport layer 5, the photoelectric conversion layer 6, the electron transport lave 7 and the cathode 8. The organic photoelectric conversion device 1 of the embodiment can absorb a light which enters the organic photoelectric conversion device 1 and can perform photoelectric conversion.

The substrate 2 is provided in order to support the other members. The material of the substrate 2 is not particularly limited as long as it is optically transmissive. Examples of this material include transparent substrates formed from glass or a synthetic resin.

The thickness of the substrate 2 is not particularly limited as long as the strength of the substrate is enough to support the other member. Also, the shape, structure and size, etc. of the substrate 2 are not particularly limited, and can be appropriately selected depending on a use application and purpose, etc.

The anode 3 is formed adjacent to the substrate 2. The anode 3 is electrically connected to the photoelectric conversion layer 6 described below, and receives the holes generated in the photoelectric conversion layer 6. The material of the anode 3 is not particularly limited as long as it is electroconductive. Examples of the material include an electroconductive metal oxide film, a semitransparent metal thin film and an organic electroconductive polymer.

Specific examples of a metal oxide film include a thin film formed from an indium oxide, a zinc oxide, a tin oxide, indium tin oxide (ITO) which is a complex of these, and a film (such as NESA) produced by using an electroconductive glass formed from fluorine-doped tin oxide (FTO). Specific examples of a metal thin film include a thin film of gold, platinum, silver or copper. Specific examples of an electroconductive polymer include polyaniline and a derivative thereof, and polythiophene and a derivative thereof. Of these, it is preferable to use a transparent electrode formed from ITO.

The thickness of the anode 3 is preferably within a range of 30 to 300 nm when using ITO. By setting the thickness of the anode 3 to 30 nm or more, it is possible to decrease the resistance of the anode 3 and to suppress a decrease in luminous efficiency due to an increase in the resistance. Also, by setting the thickness of the anode 3 to 300 nm or less, it is possible to maintain the flexibility of the anode 3 formed from ITO and to prevent cracking thereof.

The anode 3 can be a single layer or can be formed by stacking layers formed from materials having different work functions.

The planarization layer 4 is formed adjacent to the opposite side of the substrate 2 at the anode 3. Because of the planarization layer 4, it is possible to relieve the effect of the unevenness of the anode 3. The material of the planarization layer 4 is not particularly limited as long as it can relieve the effect of the unevenness of the anode 3. Specific examples thereof include polythiophene-based polymers such as poly(ethylenedioxythiophene):poly(styrenesulfonic acid) mixture (PEDOT:PSS) which is an electroconductive ink.

The hole transport layer 5 is formed between and adjacent to the planarization layer 4 and the photoelectric conversion layer 6 described below. The hole transport layer 5 prevents that the electrons are injected from the anode 3 into the side of the photoelectric conversion layer 6. Also, the hole transport layer 5 passes the holes generated in the photoelectric conversion layer 6 to the anode 3.

The material of the hole transport layer 5 is not particularly limited. Examples of the material include N,N′-bis(3-methylphenyl)-N,N′-diphenyl benzidine (TPD) and tris(4-carbazol-9-yl)phenylamine (TCTA).

The photoelectric conversion layer 6 is formed between and adjacent to the hole transport layer 5 and the electron transport layer 7 described below. The photoelectric conversion layer 6 absorbs a light having entered the organic photoelectric conversion device 1 and performs photoelectric conversion, which generates electrons and holes.

The photoelectric conversion layer 6 can be comprised of a donor material and an acceptor material. A donor material is not particularly limited, and the specific examples thereof include coumarin, quinacridone and subphthalocyanine. An acceptor material is not particularly limited, and the specific examples thereof include fullerene (C60), perylene tetracarboxydiimide and phthalocyanine.

The electron transport layer 7 is formed between and adjacent to the photoelectric conversion layer 6 and cathode 8 described below. The electron transport layer 7 prevents that the holes are injected from the cathode 8 onto the side of the photoelectric conversion layer 6. Also, the electron transport layer 7 passes the electrons generated in the photoelectric conversion layer 6 to the cathode 8.

The electron transport layer 7 is comprised of a plurality of isomers containing the compound represented by the general formula (1) and an enantiomer of the general formula (1). In other words, the electron transport layer 7 can include other isomers as long as including the compound represented by the general formula (1) and the enantiomer of the general formula (1).

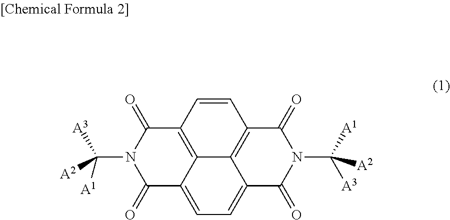

Herein, in the general formula (1), A¹, A² and A³ respectively represent a different substituent group.

Isomers are molecules in which the number of atoms, the type of atoms and the composition formulas are the same, but the bonding relationships between atoms are different. Among materials having isomeric relationship, the energy levels of respective isomers are the similar. Therefore, when the electron transport layer 7 is comprised of a plurality of compounds having isomeric relationship, it is rare to generate an electron trap, etc. attributed to the difference of the energy levels of respective isomers. Also, it is possible to suppress significant deterioration of the electron-transporting property of the electron transport layer 7.

Enantiomers are molecules in which the atomic configurations have the mirror-image relationship with each other. The electron transport layer 7 including at least two compounds having the enantiomeric relationship is made from microcrystals having a small size. Respective materials having the enantiomeric relationship have very similar structures, but are not the completely same. Therefore, when mix respective materials having the enantiomeric relationship, these materials hardly grow to a crystal having a large size.

The electron transport layer 7 of the present embodiment has the higher flatness than the electron transporting layer made from a single crystalline material. When the electron transport layer 7 is formed using a single crystalline material, a crystalline material grows to a crystal grain having a relatively large size. Because there is unevenness among crystal grains, it is difficult to enhance the flatness of the electron transport layer 7. In contrast, the electron transport layer 7 including at least two compounds having the enantiomeric relationship is made from microcrystals having a small size. When the electron transport layer 7 is made from microcrystals, unevenness among crystal grains decreases, and the flatness of the electron transport layer 7 is enhanced.

When the flatness of the electron transport layer 7 is high, it is possible to keep constant interelectrode distance between the anode 3 and the cathode 8, and also, it is possible to suppress the generation of a dark current and a locally concentrated electric field.

The electron transport layer 7 of the present embodiment has the higher heat resistance than the electron transporting layer made from an amorphous material. This is because a crystalline material generally has the higher heat resistance than an amorphous material. By enhancing the heat resistance of the electron transport layer 7, it is possible to broaden the process margin in the production of the organic semiconductor device 1 and the solid-state imaging device described below. For example, in the production process of the solid-state imaging device using a silicon photodiode, etc. it is generally to carry out a high temperature process. Because having the heat resistance, the electron transport layer 7 can withstand a high-temperature process, and there is no limitation to the production method for the organic semiconductor device 1 and the solid-state imaging device described below.

As described above, by including at least two compounds having the enantiomeric relationship in the electron transport layer 7, it is possible to balance the flatness and heat resistance of the electron transport layer 7 without significantly deteriorating the electron-transporting property.

It is preferable that the substituent groups represented by A¹, A² and A³ in the general formula (1) be selected such that the size of the dipole moment u of the compound represented by the general formula (1) falls within the range of 0≦μ<0.3. Small size of a dipole moment means small deviation of the charge throughout a compound.

When the unevenness of the charge distribution within the compound represented by the general formula (1) decreases, the relative unevenness of the charge distribution between the isomers also decreases. In other words, the difference of the dipole moments obtained when comparing the respective isomers decreases. When the difference of the dipole moments obtained when comparing the respective isomers decreases, the charge distributions of the respective isomers are similar, and the properties of the respective isomers are more similar.

When the properties of the respective isomers are more similar, it is possible to much decrease the difference of the energy levels of the materials the enantiomeric relationship. Also, it is possible to suppress that phase separation occurs in the respective isomers when forming the electron transport layer 7. When the properties of the respective isomers are different, respective deposition rates, etc. are different, and phase separation is likely to occur. Occurrence of phase separation causes the generation of polycrystals having a large grain size. Therefore, by suppressing phase separation, it is possible to enhance the flatness of the electron transport layer 7.

In the general formula (1), it is more preferable that A¹ represent hydrogen, that A² represent a methyl group, and that A³ represent an ethyl group. This compound is represented by the following general formulas (2) to (4).

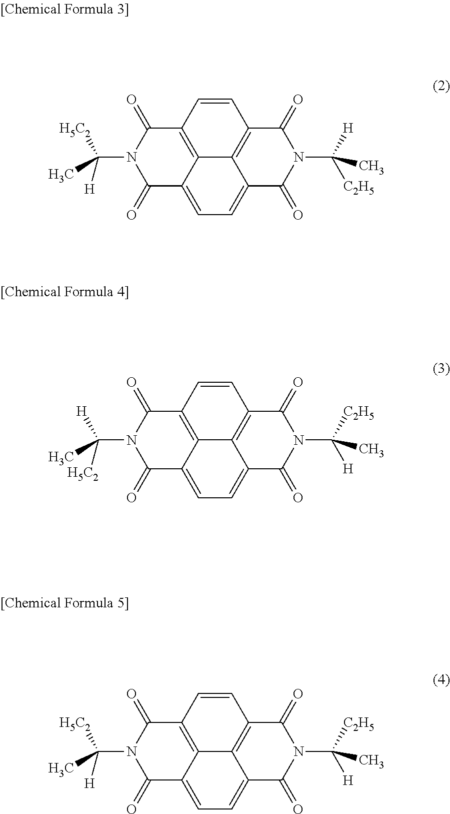

The compounds represented by the general formulas (2) to (4) have the isomeric relationship with each other. Also, the compounds represented by the general formulas (2) and (3) have the enantiomeric relationship with each other.

In the compounds represented by the general formulas (2) to (4), the respective dipole moments μ fall within the range of 0≦μ<0.3. Moreover, the molecular lengths of hydrogen and the carbon chains, which constitute A¹, A² and A³, are short. Therefore, it is possible to suppress the occurrence of the steric hindrance such as molecules, which constitute A¹, A² and A³, tangling with each other. The steric hindrance may cause the decrease in the melting point and the charge-transporting property of the charge transport layer (i.e. the electron transport layer 7).

The respective compounds represented by the general formulas (2) to (4) do not have a glass transition temperature, and have the melting point of 197° C. Therefore, the electron transport layers 7 formed from these compounds have the heat resistance to the high-temperature process of at least 150° C.

The cathode 8 is formed adjacent o the opposite side of the photoelectric conversion layer 6 at the electron transport layers 7. The cathode 8 is electrically connected to the photoelectric conversion layer 6 and receives the electrons generated in the photoelectric conversion layer 6. The material of the cathode 8 is not particularly limited as long as it is electroconductive. Specific examples thereof include an electroconductive metal oxide film, metal thin film and an alloy.

Specific examples of an alloy include a lithium-aluminum alloy, a lithium-magnesium alloy, a lithium-indium alloy, a magnesium-silver alloy, a magnesium-indium alloy, a magnesium-aluminum alloy, an indium-silver alloy, and a calcium-aluminum alloy.

The thickness of the cathode 8 is not particularly limited, but preferable examples of the thickness include a range of 10 to 150 nm. By setting the thickness to 10 nm or more, it is possible to decrease the resistance. Also, by setting the thickness to 150 nm or less, it is possible to reduce the time for the film formation and to prevent the adjacent layers from being damaged during the film formation.

The cathode 8 can be a single layer or can be formed by stacking layers made from materials having different work functions.

Next, the production method for the organic photoelectric conversion device 1 of the embodiment is described.

First, a glass substrate is prepared as the substrate 2. On the substrate 2, the transparent electroconductive film such as ITO is formed by a sputtering method as the anode 3. Examples of the film formation method for the anode 3 other than the aforementioned sputtering method include a vacuum deposition method, an ion plating method, a plating method and a coating method.

On the anode 3, an electroconductive ink such as PEDOT:PSS is applied by a method such as spin coating method as the planarization layer 4. Thereafter, the applied conductive ink is subjected to heating and drying by the hot plate, etc, so as to form a tile. As the solution to be applied, it is possible to use a solution which is preliminarily filtrated by a filter.

On the planarization layer 4, a film of a material such as TPD is formed by a vacuum deposition method as the hole transport layer 5. Examples of the film formation method for the hole transport layer 5 include a coating method in addition to the aforementioned vacuum deposition method.

On the hole transport layer 5, a film of a material such as subphthalocyanine formed by a vacuum deposition method as the photoelectric conversion layer 6. Examples of the film formation method for the photoelectric conversion layer 6 include a coating method in addition to the aforementioned vacuum deposition method.

On the photoelectric conversion layer 6, a plurality of isomers is codeposited to form the electron transport layer 7. The codeposition of a plurality of isomers is carried out in accordance with the following process.

First, the naphthalene-tetracarboxylic dianhydride represented by the general formula (5) is prepared, and the composition represented by the general formula (6) is prepared. As these compounds, it is possible to use commercially available ones.

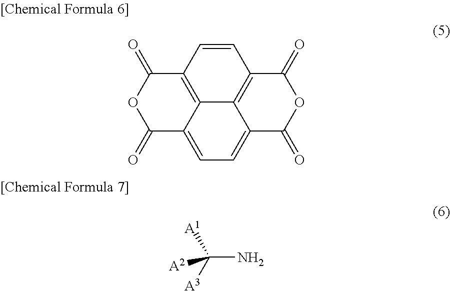

Subsequently, the compound represented by the general formula (5) is reacted with the composition represented by the general formula (6), so as to simultaneously obtain a plurality of isomers represented by the following general formulas (7) to (9) in a single synthesis. The synthesis can be carried out by heating and mixing.

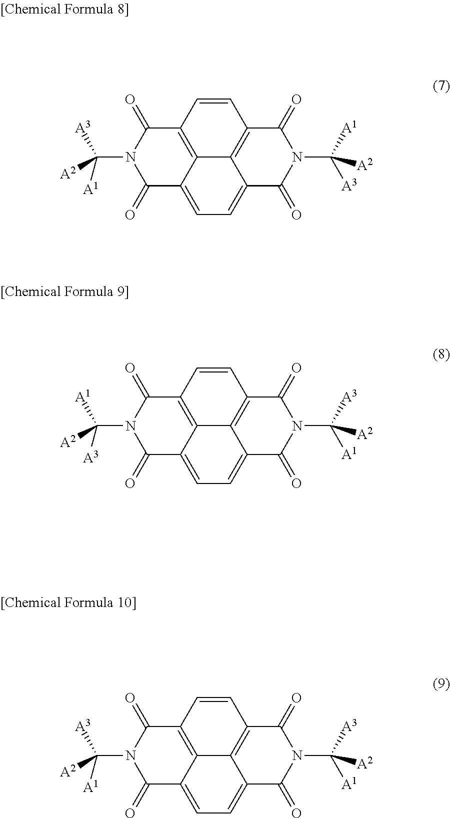

When see-butylamine is used as the composition represented by the aforementioned general formula (6), the respective compounds represented by the general formulas (7) to (9) correspond to the respective compounds represented by the general formulas (2) to (4).

The obtained plurality of isomers is simultaneously deposited under vacuum on the photoelectric conversion layer 6. It is preferable that the deposition timing of each of the plurality of isomers be the same. When the respective isomers are sequentially deposited, phase separation, etc, can occur. In order to make the deposition timing (the evaporation rate) constant, it is preferable that the respective dipole moments μ of the plurality of isomers fall within the range of 0≦μ<0.3.

On the electron transport layer 7, a film of a material such as aluminum is formed by a vacuum deposition method as the cathode 8. Examples of the film formation method for the cathode 8 include a sputtering method, an ion plating method,a plating method and a coating method in addition to the aforementioned vacuum deposition method.

Through the process described above, it is possible to produce the organic photoelectric conversion device 1 of the embodiment.

In the aforementioned embodiment, the substrate 2 is formed adjacent to the opposite side of the planarization layer 4 at the anode 3, but the e substrate 2 can be formed adjacent to the opposite side of the electron transport layer 7 at the cathode 8.

Also, in the aforementioned embodiment, the organic photoelectric conversion device 1 includes the substrate 2, but can be free from substrate 2.

Also, in the aforementioned embodiment, the organic photoelectric conversion device 1 includes the planarization layer 4 and the hole transport layer 5, but can be free from one or both of the planarization layer 4 and the hole transport layer 5.

Also, in the aforementioned embodiment, the different materials are used for the anode 3 and the cathode 8, but the same material can be used for the anode 3 and the cathode 8. In this case, it is possible to use the aforementioned materials used for the anode 3 and the cathode 8. For example, both of the materials for the anode 3 and the cathode 8 can be ITO.

According to the embodiment described above, the organic photoelectric conversion device 1 includes the electron transport layer 7 comprised of isomers containing the compound represented by the general formula (1) and the enantiomer the general formula (1). Therefore, it is possible to achieve high heat resistance and a low dark current.

### Second Embodiment

FIG. 2 is schematic diagram representing the configuration of the organic photoelectric conversion devise of the 2nd embodiment. The organic photoelectric conversion device 11 of the 2nd embodiment is the same as the organic photoelectric conversion device 1 of the 1st embodiment except that the materials forming the photoelectric conversion layer and the electron transport layer are different. Therefore, hereinafter, the materials forming the photoelectric conversion layer and the electron transport layer are described in detail, and the descriptions for common features are omitted. Also, the same reference symbols are assigned to the common features with FIG. 1 in the drawings used for the description.

The photoelectric conversion layer 16 includes a plurality of isomers containing the compound represented by the general formula (1) and the enantiomer of the general formula (1) in addition to the photoelectric conversion material. The photoelectric conversion material may be comprised of donor and acceptor materials. A donor material and an acceptor material are not particularly limited, and it is possible to use the materials exemplified in the 1st embodiment.

As described above, when forming a layer including a plurality of isomers containing the compound represented by the general formula (1) and the enantiomer of the general formula (1), it is possible to enhance the heat resistance and flatness of the formed layer. For this reason, when the photoelectric conversion layer 16 includes these isomers, it is possible to enhance the heat resistance and flatness of the photoelectric conversion layer 16. When the heat resistance of the photoelectric conversion layer 16 is high, it is possible to broaden the process margin in the production of the organic semiconductor device 1 and the solid-state imaging device described below. When the flatness of the photoelectric conversion layer 16 is high, it is possible to keep a constant interelectrode distance between the anode 3 and the cathode 8, and also, it is possible to suppress the generation of a dark current and a locally concentrated electric field.

Even in the photoelectric conversion layer 16, it is preferable that the substituent groups represented by A¹, A² and A³ in the compound represented by the general formula (1) be selected such that the size of the dipole moment μ of the compound represented by the general formula (1) falls within the range of 0≦μ<0.3. Also, it is more preferable that A¹ represent hydrogen, that A² represent a ethyl group, and that A³ represent an ethyl group.

The material of the electron transport layer 17 is not particularly limited. In addition to the materials exemplified in the 1st embodiment, t is possible to use an oxazole derivative and a triazole derivative. etc.

According to the embodiment described above, the organic photoelectric conversion device 1 includes the photoelectric conversion layer 16 comprised of isomers containing the compound represented by the general formula (1) and the enantiomer of the general formula (1). Therefore, it is possible to achieve high heat resistance and a low dark current.

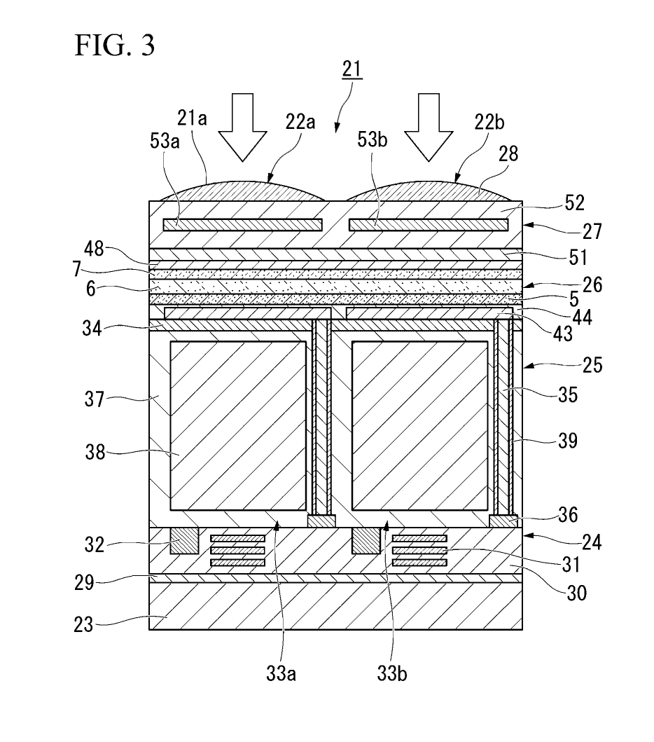

Next, the solid-state imaging device 21 of the embodiment which includes the organic photoelectric conversion device t of the embodiment s described with reference to FIG. 3. FIG. 3 is a schematic diagram representing the configuration of the solid-state imaging device 21 of the embodiment. As shown in FIG. 3, the solid-state imaging device 21 is configured to include the adjacent pixels 22a, 22b.

Only two pixels 22a, 22b are illustrated in the solid-state imaging device 21 shown in FIG. 3, but the solid-state imaging device 21 of the embodiment contains a plurality of pixels arranged in an array.

The solid-state imaging device 21 of the embodiment includes the supporting substrate 23, the wiring part 24, the 1st photoelectric conversion part 25, the 2nd photoelectric conversion part 26, the color filter part 27 and the microlens 28.

The solid-state imaging device 21 of the embodiments a back side illumination typed photoelectric conversion device. Although a back side illumination typed photoelectric conversion device is illustrated in FIG. 3 as an example, the present invention is not limited thereto, and it is possible to use a front side illumination typed photoelectric conversion device.

The supporting substrate 23 is a substrate for supporting the wiring part 24. Examples of the supporting substrate 23 include a semiconductor substrate. Also, specific examples of the semiconductor substrate include a silicon (Si) substrate,

The wiring part 24 is provided on the side of the light receiving surface 21a of the supporting substrate 23. The wiring part 24 and the supporting substrate 23 are formed through the adhesive layer 29. The wiring part 24 includes the insulating layer 30, the multilayer wiring 31 and the read transistor 32.

The insulating layer 30 is provided between and adjacent to the adhesive layer 29 and the 1st photoelectric conversion part 25. Examples of the insulating layer 30 include a silicon oxide (SiO₂).

The multilayer wiring 31 is provided respectively at the pixels 22a, 22b in the insulating layer 30, and is connected to the read transistor 32, the storage diode 36 and the peripheral circuit (not illustrated).

The multilayer wiring 31 can output the charges stored in the photodiodes 33a, 33b and the storage diode 36 to the peripheral circuit (not illustrated) as an electric signal. The material of the multilayer wiring 31 is not particularly limited as long as it is an electroconductive material. Specific examples thereof include high melting point metals such as copper (Cu), titanium (Ti), molybdenum (Mo) and tungsten (W), and high melting point metal silicides such as titanium de (TiSi) molybdenum silicide (MoSi) and tungsten silicide (WSi).

The read transistors 32 are provided at the respective pixels 22a, 22b on the surface of the wiring part 24, which is on the side of the 1st photoelectric conversion part 25. The read transistor 32 controls the movement of the charges stored in the photodiode 33a, 33b.

The 1st photoelectric conversion section 25 is provided between and adjacent to the wiring part 24 and the 2nd photoelectric conversion part 26. The 1st photoelectric conversion part 25 includes the photodiode 33a, 33b, the transparent insulating layer 34, the contact plug 35 and the storage diode 36.

The photodiodes 33a, 33b are provided in the p-type single crystal Si substrate 37 so as to correspond to the pixels 22a, 22b arranged in an array. The photodiodes 33a, 33b absorb a light of a wavelength range of one color of the three primary light colors and goes through the photoelectric conversion layer 6 described below, and perform photoelectric conversion.

Herein, the “three primary light colors” refer three colors of “a blue color”, “a green color” and “a red color”. The wavelength range of a blue light (a light of the blue wavelength range) is for example 400 to 500 nm, the wavelength range of a green light (a of the green wavelength range) is for example 500 to 600 nm, and the wavelength range of a red light (a light of the red wavelength range) is for example 600 to 700 nm.

As the photodiodes 33a, 33b, the n-type impurity diffusion region 38 is provided in the p-type single crystal Si substrate 37. The PN junction surface is formed between the p-type single crystal Si substrate 37 and the n-type impurity diffusion region 38. Herein, the photodiodes 33a, 33b are not limited to the n-type impurity diffusion region provided in the p-type single crystal Si substrate, and can be a p-type impurity diffusion region provided in an n-type single crystal Si substrate.

The p-type single crystal Si substrate is provided between and adjacent to the wiring part 24 and the transparent insulating layer 34. As the p-type single crystal Si substrate 37, for example, it is possible to use Si in which a p-type impurity such as boron has been doped. As the n-type impurity diffusion region 38, for example, it is possible to use Si in which an n-type impurity such as phosphorus has been doped.

The transparent insulating layer 34 is provided between and adjacent to the p-type single crystal Si substrate 37 and the 2nd photoelectric conversion part 26. The transparent insulation layer 34 is optically transmissive and insulates the photoelectric conversion layer 6 and the p-type single crystal Si substrate 37. Examples of the transparent insulating layer 34 include a SiO₂ film.

The contact plug 35 is provided so as to penetrate through the p-type single crystal Si substrate 37, and electrically connects the wiring part 24 and the 2nd photoelectric conversion part 26. Also, the contact plugs 35 are arranged at the respective pixels 22a, 22b so as to be positioned in a region surrounded on all four sides by the photodiodes 33a, 33b.

The contact plug 35 is electrically connected to the lower transparent electrode 43 and the storage diode 36, and can send the charges collected in the lower transparent electrode 43 to the storage diode 36. The contact plug 35 is covered with the insulating film 39. The material of the contact plug 35 is not particularly limited as long as it is an electroconductive material. Specific examples thereof include Si. Also, the insulating film 39 is not particularly limited as long as it is an insulating material. Specific examples thereof include a silicon nitride (SiN)

The storage diode 36 is provided at the end of the contact plug 35 which is on the side of the wiring part 24. The storage diode 36 temporarily stores the charges collected in the lower transparent electrode 43. A floating diffusion (not illustrated) is provided in the p-type single crystal Si substrate 37. The stored charges are sent from the storage diode 36 to the floating diffusion (not illustrated), and are converted into electric signals.

The 2nd photoelectric conversion part 26 is provided between and adjacent to the 1st photoelectric conversion part 25 and the color filter part 27. The 2nd photoelectric conversion part 26 include the lower transparent electrode 43, the planarization layer 44, the hole transport layer 5, the photoelectric conversion layer 6, the electron transport layer 7 and the upper transparent electrode 48.

In other words, the 2nd photoelectric conversion part 26 corresponds to the aforementioned organic photoelectric conversion device 1 except that the substrate 2 is omitted. Also, the anode 3 of the organic photoelectric conversion device 1 corresponds to the lower transparent electrode 43 of the 2nd photoelectric conversion section 26, and the cathode 8 of the organic photoelectric conversion device 1 corresponds to the upper transparent electrode 48 of the 2nd photoelectric conversion section 26. It is possible to use the organic photoelectric conversion device 11 of the 2nd embodiment instead of the organic photoelectric conversion device 1 of the 1st embodiment. Hereinafter, the descriptions for the corresponding parts are omitted in this specification.

The lower transparent electrodes 43 are provided at the respective pixels 22a, 22b on the surface of the transparent insulating layer 34 which is on the side of the light receiving surface 21a. Also, the peripheral part of the projection area formed by projecting the lower transparent electrode 43 to the p-type single crystal Si substrate 47 overlaps the light receiving surfaces of the photodiodes 33a, 33b in a plan view. Examples of the material of the lower transparent electrode 43 include a transparent conductive material such as indium tin oxide (ITO).

The planarization layer 44 is provided between and adjacent to the photoelectric conversion layer 6 described below, and the lower transparent electrode 43 and the transparent insulating layer 34. The planarization layer 44 can planarize the uneven surfaces of the lower transparent electrode 43 and the transparent insulating layer 34. Examples of the material of the planarization layer 44 include the same materials as the planarization layer 4 of the aforementioned organic photoelectric conversion device 1.

The upper transparent electrode 48 is provided on the surface of the photoelectric conversion layer 6, which is on the side of the light receiving surface 21a, as a single sheet so as to cover a plurality of the photodiodes 33a, 33b. Because of the upper transparent electrode 48, it is possible to apply a bias voltage supplied from the outside to the photoelectric conversion layer 6.

When applying a bias voltage, the upper transparent electrode 48 can collect the charges generated in the photoelectric conversion layer 6 in the respective lower transparent electrodes 43. Examples of the material of the upper transparent electrode 48 include a transparent conductive material such as indium tin oxide (ITO).

The color filter part 27 is provided between and adjacent to the 2nd photoelectric conversion part 26 and the microlens 28. The color filter unit 27 includes the inorganic protective film 51, the planarization layer 52, and pluralities of the 1st color filter 53a and the 2nd color filter 53b.

The inorganic protective film 5 l is provided on the surface of the upper transparent electrode 48, which is on the side of the light receiving surface 21a, as a single sheet. Examples of the inorganic protective film 51 include an aluminum oxide (Al₂O3) film.

The planarization layer 52 is provided between and adjacent to the 2nd photoelectric conversion section 26 and the microlens 28. Examples of the material of the planarization layer 52 include silicon dioxide.

The pluralities of the 1st color filter a and the 2nd color filter 53b are provided in the planarization layer 52 so as to face the photodiodes 33a, 33b. The 1st color filter 53a absorbs a light of a specific wavelength range and is transmissive to a light of other wavelength ranges. Also, the 2nd color filter 53b can be the same as the 1st color filter 53a, and can be a different color filter which absorbs a light of other wavelength ranges.

For example, the 1st color filter 53a can be configured to absorb a blue light and to be transmissive to a green light and a red light, and the 2nd color filter 53b can be configured to absorb a red light and to be transmissive to a blue light and a green light.

By appropriately selecting the wavelength ranges of lights absorbed by the 1st color filter 53a and the 2nd color filter 53b, it is possible to select the wavelength range of a light absorbed by the photoelectric conversion layer 6.

The microlenses 28 are provided on the side of the light receiving surface 21a of the color filter portion nd at the positions which face the photodiodes 33a, 33b. For example, the microlens 28 can be a lens which forms a circle in planer view such that incident light is focused by the microlens 28. The optical centers of the respective microlenses 28 are positioned at the centers of the light receiving surfaces of the respective photodiodes 33a, 33b. The plan-view area of the microlens 28 is larger than the area of the light receiving surface of the photodiodes 33a, 33b.

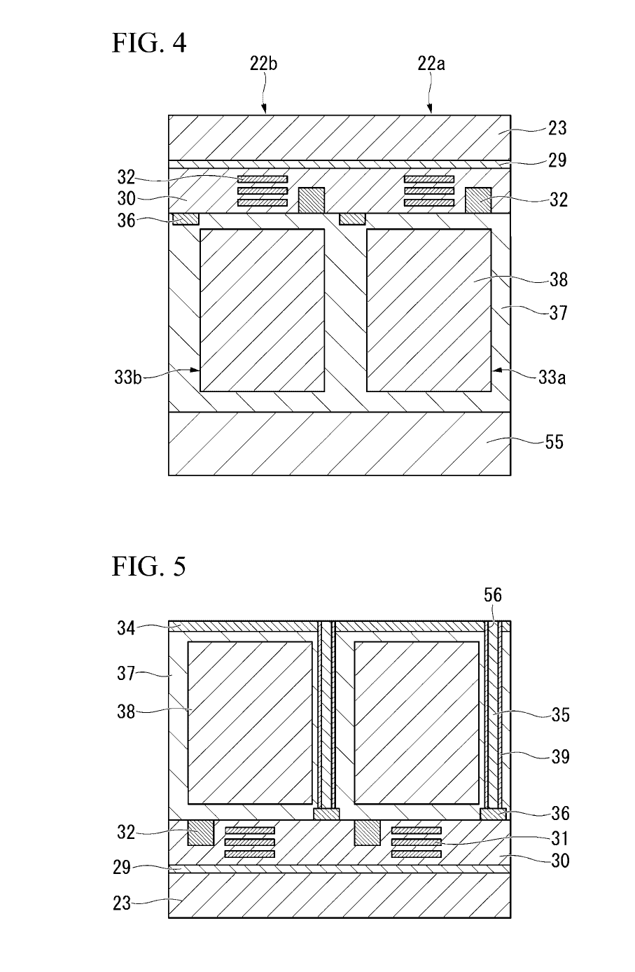

Next, the production method for the solid-state imaging device 21 of the embodiment is described with reference to FIG. 4 to FIG. 7. FIG. 4 to FIG. 7 are the schematic diagrams representing the production method for the solid-state imaging device 21 of the embodiment.

First, as shown in FIG. 4, the p-type single crystal Si substrate 37 is formed by epitaxially growing the Si layer, in which a p-type impurity such as boron has been doped, on the semiconductor substrate 55 such as a Si wafer.

The n-type impurity diffusion region 38 is set in the respective pixels 22a, 22b within the p-type single crystal Si substrate 37. For example, the n-type impurity diffusion region 38 can be obtained by subjecting the respective pixels 22a, 22b within the p-type single crystal Si substrate 37 to the ion-implantation using an n-type impurity such as phosphorus and an annealing treatment. Through this process, the photodiodes 33a, 33b are formed in the solid-state imaging device 21 by the PN junction between the p-type single crystal Si substrate 37 and the n-type impurity diffusion region 38.

The other n-type impurity diffusion region such as the storage diode 36 is formed in the inner surface of the p-type single crystal Si substrate 37. For example, the storage diode 36 is obtained by subjecting the inner surface of the p-type single crystal Si substrate 37 to the ion-implantation using an n-type impurity such as phosphorus and an annealing treatment. If necessary, it is possible to form the pixel isolation region, etc. (not illustrated) by further subjecting the inner surface of the p-type single crystal Si substrate 37 to the ion-implantation using an p-type impurity such as boron and an annealing treatment.

The insulating layer 30 is formed on the p-type single crystal Si substrate 37 together with the multilayer wiring 31 and the read transistor 32. Specifically, the read transistor 32, etc. is formed on the upper surface of the p-type single crystal Si substrate 37, followed by repeating the step of forming the Si oxide layer, the step of forming a predetermined wiring pattern on the Si oxide layer, and the step of embedding Cu, etc. within the wiring pattern. This process forms the insulating layer 30 provided with the multilayer wiring 31 and the read transistor 32, etc.

An adhesive is applied onto the upper surface of the insulating layer 30, to thereby form the adhesive layer 29. Then, the supporting substrate 23 such as a Si wafer is attached onto the upper surface of the adhesive layer 29 The attachment of the insulating layer 30 and the supporting substrate 23 is not limited to the attachment using an adhesive, but the direct attachment of the insulating layer 30 and the supporting substrate 23 is also possible by subjecting the insulating layer 30 to the polishing such as CMP (Chemical Mechanical Polishing) so as to prepare a flat and smooth surface thereof.

The surface of the Si wafer including the photodiodes 33a, 33b, which is on the opposite side to the supporting substrate 23, is ground by a grinding apparatus such as a grinder, to thereby reduce the thickness of the Si wafer to a predetermined thickness. Then, the surface of the semiconductor substrate is polished by a polishing apparatus such as a CMP apparatus, and moreover, is subjected to wet etching, etc., to thereby remove the damaged layer of the surface of the semiconductor substrate. This process exposes the light-receiving surface of the p-type single crystal Si substrate 37.

Subsequently, as shown in FIG. 5, the transparent insulating layer 34 made from a transparent insulating material such as SiO₂ is formed on the upper surface of the p-type single crystal Si substrate 17.

The positions surrounded on all four sides by the respective photodiodes in the formed transparent insulating layer 34 and the p-type single crystal Si substrate 37 are removed by RIE (Reactive Ion Etching), etc. until reaching the top of the storage diode 36. This process forms the trenches 56. On the inner surface of the trench 56, the insulating film 39 made from an insulating material such as SiN is formed by a CVD (Chemical Vapor Deposition) method, etc.

Within the trenches 56 having the surface coated with the insulating film 39, the contact plugs 35 formed from an electroconductive material such as Si are embedded by a CVD method, etc. The embedding method of the contact plug 35 is not limited to the aforementioned method. Before and after the step of forming the impurity diffusion region such as the storage diode 36, it is possible to form the other n-type impurity diffusion region as the contact plug 35 by subjecting the inner surface of the p-type single crystal Si substrate 37 to the on-implantation using an n-type impurity such as phosphorus and an annealing treatment.

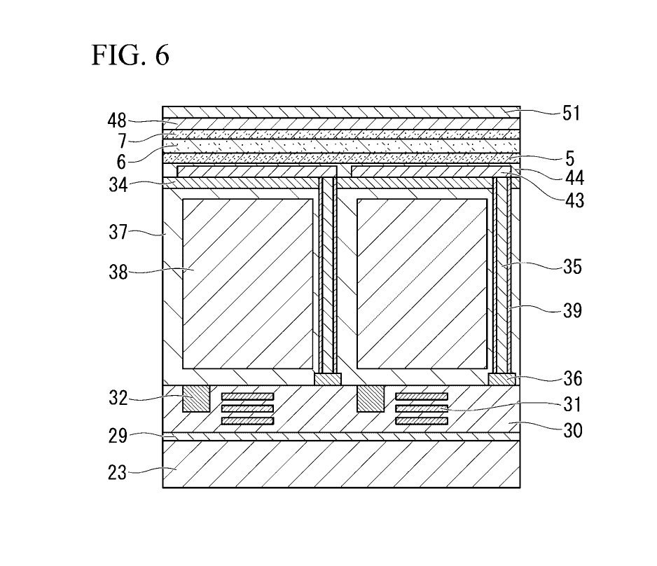

Subsequently, as shown in FIG. 6, the lower transparent electrode 43 made from a transparent conductive material such as ITO is formed on the upper surface of the transparent insulating layer 34 and the upper surfaces of the exposed contact plugs 35. The lower transparent electrode 43 can be formed in a predetermined shape by using photolithography.

After the for ation of the lower transparent electrode 43, a transparent resin is applied on the lower transparent electrode 43 and the transparent insulating layer 34 by a coating process such as a spin coating method. Through this process, it is possible to form the planarization layer 44. Thereafter, a film made from TPD (N,N′-diphenyl-N,N′-di(m-tolyl)benzidine), etc. is formed on the upper surface of the planarization layer 44 by a vacuum deposition method, to thereby form the hole transport layer 5.

On the upper surface of the hole transport layer 5, the photoelectric conversion layer 6 and the electron transport layer 7 are formed by a vacuum deposition method, etc. When the photoelectric conversion layer 6, the electron transport layer 7, or both of the photoelectric conversion layer 6 and the electron transport layer 7 contain a plurality of isomers, the photoelectric conversion layer 6 and the electron transport layer 7 are formed by using codeposition.

On the upper surface of the hole-blocking layer 7, the upper transparent electrode 48 made from a transparent electroconductive material such as ITO is formed by a sputtering method, etc. Thereafter, on the upper surface of the upper transparent electrode 48, an Al₂O₃ film is formed as the inorganic protective film 51 by a sputtering method, etc.

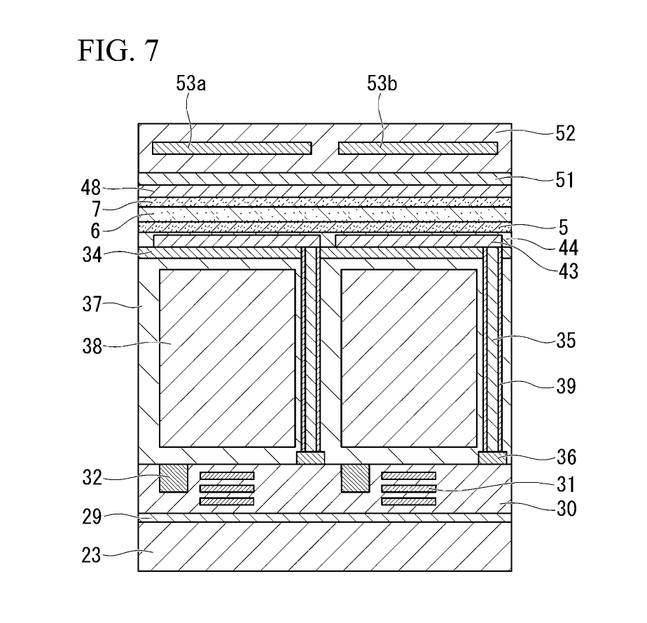

Subsequently, on the inorganic protective film 51, the planarization layer 52 made from a transparent resin is formed as shown in FIG. 7. The 1st and 2nd color filters 53a, 53b are formed at the positions, which respectively face the light-receiving surfaces of the respective photodiodes 33a, 33b in the planarization layer 52, by photolithography using a pigment or a dye for color filters which are transmissive to a green light and a red light. Then, the planarization layer 52 made from a transparent resin is further formed so as to cover the 1st and 2nd color filters 53a, 53b. Through this process, the 1st and 2nd color filters 53a, 53b are embedded in the planarization layer 52.

Finally, the microlenses 28 made of an acrylic organic compound, etc. are formed on the upper surface of the planarization layer 52 and at the positions, which respectively face the light-receiving surfaces of the respective photodiodes 33a, 33b, in a size to cover the light receiving surfaces in planer view. Through the aforementioned process, the solid-state imaging device 21 of the embodiment is produced.

In the aforementioned embodiment, the solid-state image device 21 includes the planarization layer 4 and the hole transport layer 5, but can be free from any one or both of the planarization layer 4 and the hole transport layer 5.

According to the embodiment described above, the solid-state imaging device 21 includes the layer comprised of isomers containing the compound represented by the general formula (1) and the enantiomer of the general formula (1). Therefore, it is possible to achieve high heat resistance and a low dark current.

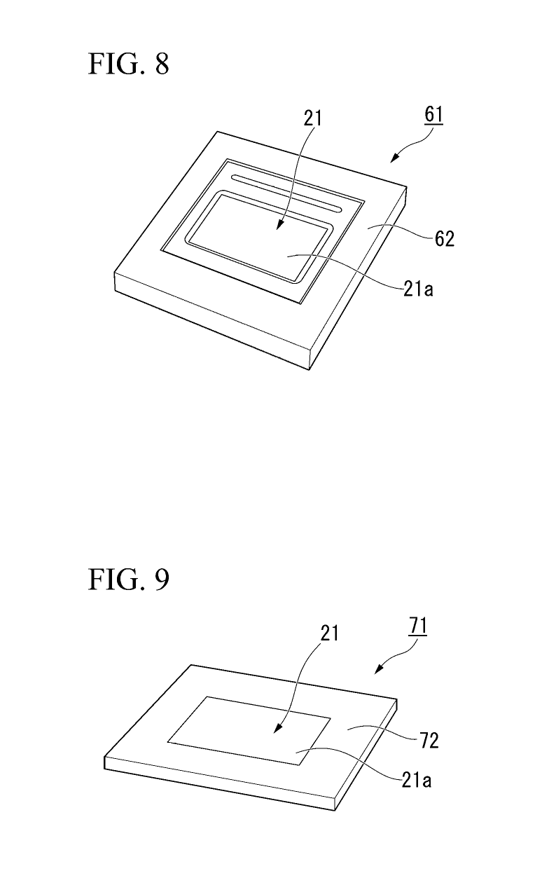

FIG. 8 is a perspective view showing an example of the CMOS image sensor 61 using the solid-state imaging device 21 of the embodiment. The CMOS image sensor 61 is a CMOS image sensor of Full HD (1080p) type. The CMOS image sensor 61 includes the solid-state imaging device 21 and the mold resin 62.

The mold resin 62 is provided so as to cover the part other than the light receiving surface 21a of the solid-state imaging device 21. By integrating the solid-state imaging device 21 and the mold resin 62, it is possible to protect the solid-state imaging device 21 from external stress, moisture and contaminants.

The CMOS image sensor 61 is used in various mobile terminals such as a digital camera and a cellular phone (including a smartphone), a security camera, and an imaging device such as a web camera using the Internet.

FIG. 9 is a perspective view showing another example of the CMOS image sensor using the solid-state imaging device 21 of the embodiment. The CMOS image sensor 71 is a CMOS image sensor of VGA type. The CMOS image sensor 71 includes the solid-state imaging device 21 and the mold resin 72.

The mold resin 72 is provided so as to cover the part other than the light receiving surface 21a of the solid-state imaging device 21. By integrating the solid-state imaging device 21 and the mold resin 72, it is possible to protect the solid-state imaging device 21 from external stress, moisture and contaminants.

The CMOS image sensor 71 is used in various mobile terminals such as a digital camera and a cellular phone (including a smart phone), a security camera, and an imaging device such as a web camera using the Internet.

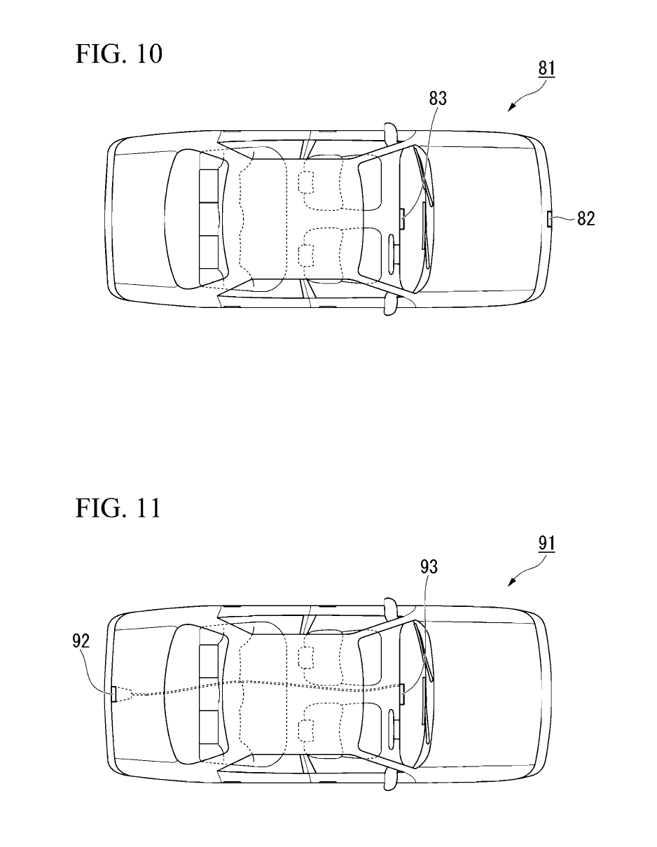

FIG. 10 is a plan view showing an example of the vehicle 81 including the camera 82 equipped with the aforementioned CMOS image sensor 61 or CMOS image sensor 71. The vehicle 81 includes the camera 82 and the display 83. The camera 82 is provided at the forward end of the vehicle 81, and it is possible to shoot the front of the vehicle 81. Also, the display 83 is provided in the front of the driver's seat of the vehicle 81, and it is possible to show the images shot by the camera 82. By checking the images shot by the camera 82 on the display 83, it is possible to check blind spots during parking, etc.

FIG. 11 is a plan view showing another example of the vehicle 91 including the camera 92 equipped with the aforementioned CMOS image sensor 61 or CMOS image sensor 71. The vehicle 91 includes the camera 92 and the display 93. The camera 92 is provided at the back end of the vehicle 91, and it is possible to shoot the back of the vehicle 91. Also, the display 93 is provided in the front of the driver's seat of the vehicle 91, and it is possible to show the images shot by the camera 92. By checking the images shot by the camera 92 on the display 93, it is possible to check the back.

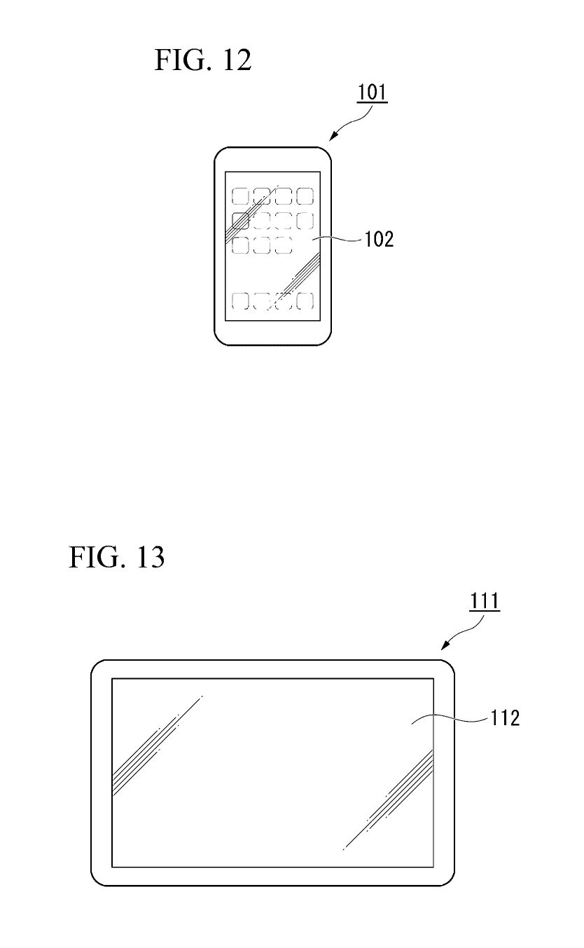

FIG. 12 is a plan view showing the smartphone 101 including the camera equipped with the aforementioned CMOS image sensor 61 or CMOS image sensor 71. The smartphone 101 includes a camera (not illustrated) and the touch panel 102. When a camera is provided at the front upper part of the smartphone 101, it is possible to shoot the front of the smartphone 101. Also, the touch panel 102 is provided in the center of the front of the smartphone, and it is possible to show the images shot by a camera.

FIG. 13 is a plan view showing the tablet 111 including the camera equipped the aforementioned CMOS image sensor 61 or CMOS image sensor 71. The tablet 111 includes a camera (not illustrated) and the touch panel 112. When a camera is provided at the front upper part of the tablet 111, it is possible to shoot the front of the tablet 111. Also, the touch panel 112 is provided in the center of the front of the tablet, and it is possible to show the images shot by a camera.

### EXAMPLES

Hereinafter, Example 1 is described.

First, naphthalene tetracarboxylic dianhydride (NTCDA) manufactured by Sigma-Aldrich Co. LLC. and sec-butylamine manufactured by Tokyo Chemical Industry Co., Ltd. were prepared.

Subsequently, in an argon atmosphere, NTCDA 50 g, sec-butylamine 41 g and Zn(OAc)₂ 41 g were added in dehydrated N-methyl-2-pyrrolidone 1.5 liters, and were reacted for 24 hours at 155° C. After cooling, 3 liters of water was added to the reaction solution. Then, the precipitated solid was collected by filtration, and washed with water and methanol. The obtained crude product was purified with silica gel column with the mixed solvent of toluene and ethyl acetate and recrystallization from toluene. The three isomers represented by the general formulas (2) to (4) were obtained as the reaction product.

The melting points and glass transition temperatures of the obtained three isomers were measured by using DSC (differential scanning calorimetry) and TG-DTA (thermogravimetric-differential thermal measurement). As a result, the glass transition temperatures were not observed, and the melting points were 197° C.

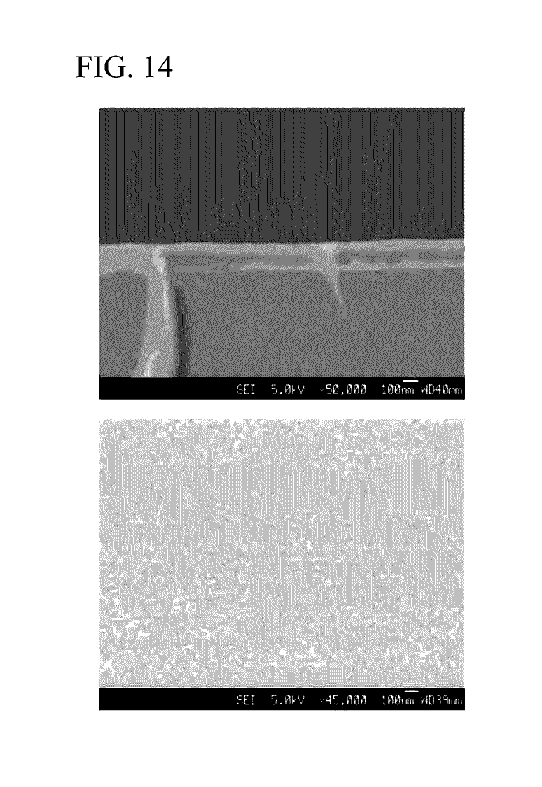

Subsequently, the obtained three isomers were codeposited on the surface of the glass substrate having an ITO film, on which an ITO film was formed, in the vacuum deposition apparatus under the condition of the deposition rate of 0.5 A/s, FIG. 14 is the plan and cross-sectional views obtained by taking photographs of the surface of the film, which was formed from the compounds having three isomers obtained by reacting sec-butylamine and NTCDA, with a scanning electron microscope (SEM).

Hereinafter, Comparative Example 1 is described.

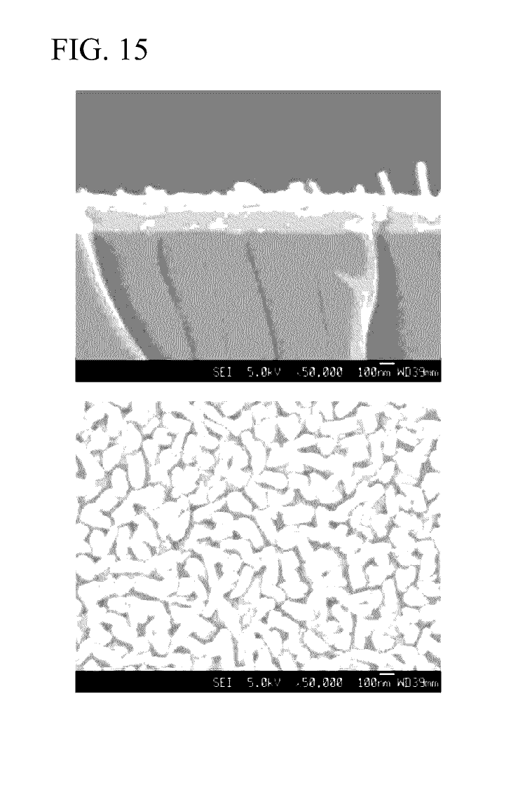

In Comparative Example 1, NTCDA was prepared, and deposited on the surface of the glass film having an ITO film on which an ITO film was formed. FIG. 15 is plan and cross-sectional views obtained by taking photographs of the surface of the film formed from NTCDA alone with a scanning electron microscope (SEM). The glass transition temperature of NTCDA was not observed, and the melting point thereof was 270° C. or more.

When comparing the image of FIG. 14 (Example 1) with the image of FIG. 15 (Comparative Example 1), it was found that the surface of the film formed from the compounds having three isomers obtained by reacting sec-butylamine and NTCDA was flattened. In other words, when the electron transport layer and the photoelectric conversion layer were formed from the materials of Example 1, it was possible to enhance the flatness of the electron transport layer and the photoelectric conversion layer, and it was possible to suppress the generation of a dark current.

Subsequently, the dipole moment, HOMO (Highest Occupied Molecular Orbital) level and LUMO (Lowest Unoccupied Molecular Orbital) level of the molecule were measured by simulation while rotating the sec-butyl group about the N—C bond of the isomers represented by the general formulas (2) to (4) as the central axis. The simulation was carried out by using a conventional method. Specifically, the simulation was carried out by using the calculation method: B3LYP and the basis function: 6−31++G (d, p).

In consideration of the isomers and the rotational angle about the N—C bond as the central axis, the 12 types of calculations were carried out. Specifically explaining the 12 kinds of calculations, the 6 simulations of the molecule, in which the sec-butyl group bonded to the 2nd end part was rotated by 60° about the sec-butyl group bonded to the 1st end part, were carried out for the respective the general formulas (2) and (4). The simulation results of the general formula (2) are shown in Table 1, and the simulation results of the general formula (4) are shown in Table 2.

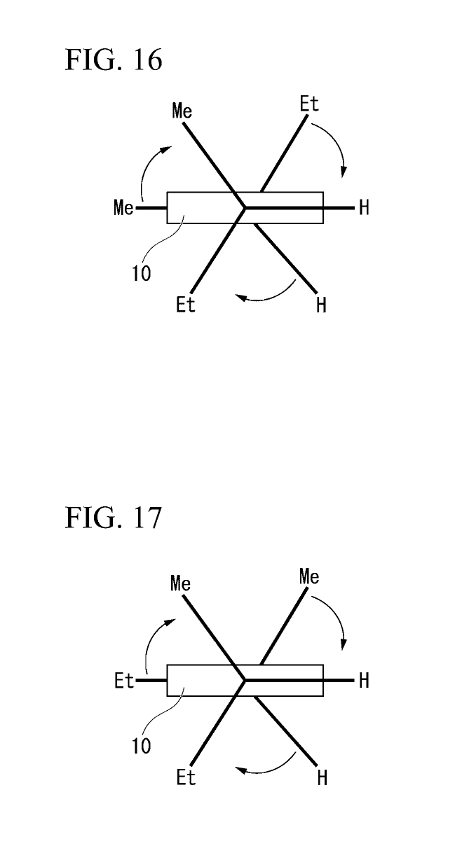

FIG. 16 and FIG. 17 are the diagrams for explaining a rotational direction of the molecular structure in the simulation. FIG. 16 is the schematic diagram obtained when observing the molecular structure represented by the general formula (2) from the side of the 1st end part, and FIG. 17 is the schematic diagram obtained when observing the molecular structure represented by the general formula (4) from the side of the 1st end part. The 1st end parts correspond to the left end parts of the molecules represented by the general formula (2) and the general formula (4). The configurations shown in FIG. 16 and FIG. 17 were defined as the rotational angle of 0°, and the clockwise direction was defined as the positive rotational direction, and the counterclockwise direction was defined as the negative rotational direction. In FIG. 16 and FIG. 17, the rectangular blocks are schematically described as the molecular main structures 10. Also, the molecular structure represented by the general formula (3) has the enantiomeric relationship with the molecular structure represented by the general formula (2) at respective rotational angles, and the dipole moments thereof are the same. Therefore, the calculation for the general formula (3) was omitted.

TABLE 1

| Rotational | Dipole Moment μ | | |
|---|---|---|---|
| Angle (°) | (debye) | HOMO Level (eV) | LUMO Level (eV) |
| 120 | 0.0897 | −7.22434 | −3.66945 |
| 60 | 0.0901 | −7.22434 | −3.66945 |
| 0 | 0.2516 | −7.22434 | −3.66945 |
| −60 | 0.2508 | −7.22434 | −3.66945 |
| −120 | 0.2527 | −7.22434 | −3.66972 |
| −180 | 0.0892 | −7.22434 | −3.66945 |

TABLE 2

| Rotational | Dipole Moment μ | | |
|---|---|---|---|
| Angle (°) | (debye) | HOMO Level (eV) | LUMO Level (eV) |
| 120 | 0.0004 | −7.22434 | −3.66945 |
| 60 | 0.0012 | −7.22407 | −3.66945 |
| 0 | 0.2744 | −7.22407 | −3.66972 |
| −60 | 0.2755 | −7.22407 | −3.66972 |
| −120 | 0.2753 | −7.22407 | −3.66972 |
| −180 | 0.0014 | −7.22434 | −3.66945 |

The sec-butyl group has the less unevenness of the charge distribution, and as shown in Table 1 and Table 2, the sizes of the dipole moments of the molecules are 0.3 or less in any cases. As a result, it was found that the HOMO-LUMO levels were approximately the same in any case. In other words, the respective isomers represented by the general formulas (2) to (4) have approximately the same HOMO-LUMO level. In other words, even when these isomers are mixed in the electron transport layer and the light-emitting layer, the electron is hardly trapped by these level differences, and the electron-transporting property is not deteriorated.

While certain embodiments have been described, these embodiments have been presented by way of example only, and are note intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An organic photoelectric conversion devise comprising:
an electron transport layer comprised of a plurality of isomers containing a compound represented by a following general formula (1) and an enantiomer of the following general formula (1). 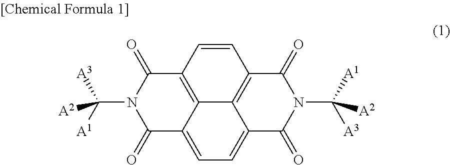
In the general formula (1), A¹, A² and A³ respectively represent a different substituent group.

2. An organic photoelectric conversion devise comprising:
a photoelectric conversion layer comprised of a plurality of isomers containing a compound represented by a following general formula (1) and an enantiomer of the following general formula (1). 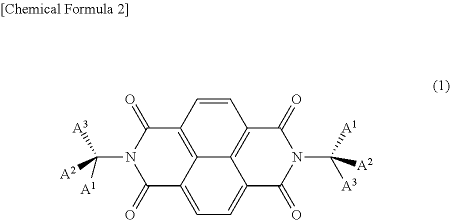
In the general formula (1), A¹, A² and A³ respectively represent a different substituent group.

3. The organic photoelectric conversion devise according to claim 1, wherein
the substituent groups represented by A¹, A² and A³ are selected such that a size of a dipole moment μ of the compound represented by the general formula (1) falls within the range of 0≦μ<0.3.

4. The organic photoelectric conversion devise according to claim 1, wherein
A¹ represents hydrogen, A² represents a methyl group, and A³ represents an ethyl group.

5. A solid-state imaging device comprising the organic photoelectric conversion devise according to claim 1.

6. The organic photoelectric conversion devise according to claim 2, wherein
the substituent groups represented by A¹, A² and A³ are selected such that a size of a dipole moment μ of the compound represented by the general formula (1) falls within the range of 0≦μ<0.3.

7. The organic photoelectric conversion devise according to claim 2, wherein
A¹ represents hydrogen, A² represents a methyl group, and A³ represents an ethyl group.

8. A solid-state imaging device comprising the organic photoelectric conversion devise according to claim 2.
